(19) United States
(12) Patent Application Publication
(10) Pub. No.: US 2008/0145425 A1
(43) Pub. Date: Jun. 19, 2008

(54) PHARMACEUTICAL COMPOSITION OF ZOLPIDEM

(75) Inventors: Leljak Marija, Durmanec (HR); Miric Snjezana, Zagreb (HR); Zvonimir Siljkovic, Zagreb (HR); Ernest Mestrovic, Bjelovar (HR)

Correspondence Address: RISSMAN JOBSE HENDRICKS & OLIVERIO, LLP, 100 Cambridge Street, Suite 2101, BOSTON, MA, 02114, US

(73) Assignee: Pliva Research & Development Limited, Zagreb (HR)

(21) Appl. No.: 11/611,305

(22) Filed: Dec. 15, 2006

(51) Int. Cl. A61K 31/437; A61K 9/32; A61P 25/00

(52) U.S. Cl. 424/482; 514/300

(57) ABSTRACT

A pharmaceutical composition comprising a Zolpidem hemitartrate starting material comprised of form A, the composition containing less than about 8% by weight of water. The starting material typically comprises less than about 0.1% by weight of forms of Zolpidem other than Form A.

### BACKGROUND

Zolpidem hemitartrate and its various polymorphs are known molecules. Isolation of certain polymorphs and preparation of compositions that retain/contain one or more of such polymorphs of Zolpidem hemitrate in stable form for use in treating select medical conditions can be problematic.

### SUMMARY OF THE INVENTION

The present invention relates to a pharmaceutical composition comprising polymorphic Form A of N,N-6-trimethyl-2-p-toyl-imidazo(1,2,-a)pyridine-3-acetamide L-(+)-tartrate herein after referred to as Zolpidem, or a pharmaceutically acceptable salt thereof (preferably tartrate), and for the use of such compositions for treating select conditions, symptoms, diseases or disorders such as insomnia. Zolpidem tartrate is also known as Zolpidem hemitartrate which is the actual form of the compound, a salt comprising two drug molecules and one tartaric acid molecule as is known in the art. The present invention also provides a manufacturing process for the preparation of a Zolpidem tartrate pharmaceutical composition made from and retaining polymorphic Form A of Zolpidem tartrate, the composition being formed into an administrable form as a tablet, capsule or granules. The preferred form of the pharmaceutical composition is tablet preferably having a film coating, i.e. a film coated tablet. A pharmaceutical composition according to the invention can include selected excipients that ensure adequate flowability of a dry blend as well as required content uniformity and drug release rate of the final product.

Insomnia is defined as sleep problems characterized by difficulty falling asleep, frequent wakings during the night, or waking up earlier than desired. Insomnia results in difficulty in initiating and/or maintaining sleep. It is a term that is used often to indicate any and all stages and types of sleep loss. Insomnia is a common side-effect of some medications, and it can also be caused by stress, emotional upheaval, physical or mental illness, dietary allergy and poor sleep hygiene. Insomnia is not a disorder, it is a symptom. Zolpidem tartrate belongs to the group of medicines called central nervous system (CNS) depressants (medicines that slow down the nervous system). In general, when sleep medicines are used every night for a long time, they may lose their effectiveness. Insomnia can affect not only energy level and mood, but a subject's general health level as well because sleep helps bolster the immune system. Insomnia may be temporary or chronic. In most cases, sleep medicines should be used only for short periods of time, such as 1 or 2 days, and generally for no longer than 1 or 2 weeks.

In accordance with the invention there is provided a pharmaceutical composition comprising a Zolpidem hemitartrate starting material comprised of form A, the composition containing less than about 8% by weight of water. The composition contains less than about 1% by weight of forms of Zolpidem tartate other than form A. The is formed in a water free environment from a powdered starting material having a maximum particle size of less than about 300 μm. The composition is preferably formed in a water free environment into a tablet from a powdered starting material by application of compression force of less than about 20 kN to the powdered starting material. The powdered starting material preferably has a maximum particle size of less than about 300 μm.

The Zolpidem hemitartrate starting material contains between about 2% and about 3.5% by weight of water and contains less than about 0.1% by weight of forms of Zolpidem hemitartrate other than Form A. The composition contains less than about 1% by weight of forms of Zolpidem hemitartrate other than Form A, preferably less than about 0.5% and most preferably less than about 0.1%. The composition preferably comprises from about 50% to about 92% by weight of a filler comprised of one or more of lactose monohydrate, lactose anhydrate, starch, sugar, sugar alcohols and celluloses. Typically, the composition comprises from about 1% to about 10% of a binder material that is comprised of one or more of hydroxypropyl cellulose, hydroxypropylmethyl cellulose, cellulose ethers and vinylpyrrolidone containing polymers. Preferably, the composition comprises from about 50% to about 97% by weight of the filler material and the binder material combined.

Typically a composition according to the invention has an X-Ray powder diffraction pattern having major peaks at 6.5, 9.0 and 16.6±0.2 degrees two-theta.

Where a composition according to the invention is formed into a tablet, the tablet is typically coated with a film comprising a polymer. The film or coating polymer preferably comprises one or more of hydroxypropylmethyl cellulose, hydroxypropyl cellulose, methylcellulose and polyvinyl alcohol. The film typically further comprises a plasticizer. The plasticizer typically comprises one or more of glycerin, propylene glycol and polyethylene glycol. The film typically further comprises a vehicle for the components of the film such as water. Preferably the film further comprises a coloring agent.

Preferably, a composition according to the invention further comprises a wetting agent in amount of between about 0.3% and about 1% by weight of the composition. A composition according to the invention can further comprises an anti-adherent material in amount of between about 0.1% and about 5% by weight of the composition, a disintegrant material in amount of between about 1% and about 10% by weight of the composition and a lubricant material in amount of between about 0.1% and about 5% by weight of the composition.

The powdered material of which one composition according to the invention is comprised is formed in a water free environment into granules having a maximum particle size of less than about 1200 μm.

Further in accordance with the invention there is provided a pharmaceutical composition comprising a Zolpidem hemitartrate starting material comprised of form A, the composition containing less than about 1% by weight of forms of Zolpidem tartate other than form A. Such a composition preferably contains less than about 8% by weight of water and is formed in a water free environment into a tablet from a powdered starting material by application of compression force of less than about 20 kN to the powdered starting material.

In another aspect of the invention, there is provided a pharmaceutical composition comprising a Zolpidem hemitartrate starting material comprised of form A, the composition being formed in a water free environment into a tablet from a powdered starting material by application of compression force of less than about 20 kN to the powdered starting material. Such a composition preferably contains less than about 8% by weight of water and less than about 1% by weight of forms of Zolpidem hemitartate other than form A.

Futher in accordance with the invention there is provided a pharmaceutical composition comprising a Zolpidem hemitartrate starting material comprised of form A, the composition being formed into a powdered material, the powdered material being formed in a water free environment into granules having a maximum particle size of less than about 1200 μm.

In another aspect of the invention there is provided, a pharmaceutical composition formed from a Zolpidem hemitartate starting material comprised of form A that is comprised of between about 2% and about 3.5% water. Such a composition preferably comprises less than about 8% by weight of water and less than about 1% by weight of forms of Zolpidem hemitartrate other than form A. Such compositions are preferably formed in a water free environment into a tablet from a powdered starting material by application of compression force of less than about 20 kN to the powdered starting material.

In another aspect of the invention there is provided, a pharmaceutical composition formed from a Zolpidem hemitartate starting material that is comprised of form A and less than about 0.1% by weight of forms of Zolpidem hemitartrate other than Form A.

Futher in accordance with the invention there is provided a method of forming a pharmaceutical composition comprising stable Form A Zolpidem hemitartrate, the method comprising the steps of:
forming a composition containing the Form A into a powdered starting material;
compressing the powdered starting material in a water free environment into a tablet by application of a pressure of less than about 20 kN to the powdered starting material.

The step of forming typically comprises homogenizing a blend of the Form A and selected excipients to obtain tableting blend having a particle size of up to a maximum of about 300 μm. Such a method further typically comprises coating the tablet with a film comprising a polymer.

Further in accordance with the invention there is provided a method of forming a pharmaceutical composition comprising stable Form A Zolpidem hemitartrate, the method comprising the steps of:
forming a composition containing the Form A into a powdered starting material;
converting the powdered starting material in a water free environment into granules.

Such a method preferably futher comprises compressing the granules in a water free environment into a tablet by application of a pressure of less than about 20 kN to the granules. The powdered starting material preferably has a particle size of up to a maximum of about 300 μm. The granules preferably have a particle size of up to a maximum of about 1200 μm. Such a method can alternatively comprise encapsulating the granules in digestable capsules. Such a method can further comprise the step of coating the tablet with a film comprising a polymer.

### DETAILED DESCRIPTION OF THE INVENTION

The present invention provides a pharmaceutical composition containing the labile polymorphic Form A of Zolpidem tartrate and a water free manufacturing process with low energy input for manufacturing tablets containing Zolpidem tartrate Form A from a synthesized or purified batch of relatively pure Zolpidem tartrate Form A. Polymorphic forms are different crystalline forms of the same chemical compound. Less stable polymorphic forms can usually be converted to more stable forms by heat, recrystallization, stirring in a solvent or sometimes by mechanical mixing or grinding. The least stable forms of one compound are normally lower melting than its more stable forms. The most stable forms usually have the highest melting points. A water free environment or process of manufacture is one where water is not present in or added to the composition of Zolpidem plus excipients. Such water free processes of formulation can be conducted in an atmospheric environment of less than about 60% relative humidity, typically between about 25% and about 60% relative humidity. With respect to a Zolpidem/excipient tablet that is coated with a film, the coating material may be formed as a suspension in water before being coated onto the Zolpidem, such process still being considered water free as used herein.

Polymorphic forms of Zolpidem hemitartrate including, A through H and L can be characterized in a variety of ways including via X-ray powder diffraction (XRPD), differential scanning calorimetry (DSC) and/or thermal gravimetric analysis (TGA). XRPD is a form of X-ray diffraction that is commonly used to identify different polymorphic forms by the pattern of X-rays diffracted by randomly oriented, finely powdered crystals. The sample of fine powder is usually rotated to insure randomness of the micro-crystals in the sample. The pattern of the diffraction angles observed (designated by the angle two-theta) and the amplitude of the signal at each two-theta serve as a usually unique fingerprint of a pure polymorph of one chemical compound. Although it is possible to extract more detailed information about the crystal structure of the polymorph from the pattern, XRPD is usually used only for fingerprint identification of specific polymorphs of organic compounds. For a brief discussion of XRPD for organic compounds see USP 24 (2000) <941> X-Ray Diffraction.

Form A is an unstable polymorph relative to other polymorphs of Zolpidem tartrate. Form A is convertable to other forms when subject to various conditions as evidenced in WO 01/80857, the disclosure of which is incorporated herein by reference in its entirety, showing the appearance of new peaks, i.e. new forms, in an XRPD scan of a micronized sample of Form A. Common manufacturing processes such as wet granulation can routinely convert the labile Form A to other more stable Forms. It has surprisingly been found that certain water free manufacturing methods for producing tablets with low energy input prevent the undesired conversion of the Form A of Zolpidem tartrate to other Forms and therefore achieve the desired stability of the final pharmaceutical composition of Form A. Accordingly there is provided according to the present invention a pharmaceutical composition comprising Zolpidem or a pharmaceutically acceptable salt (preferably tartrate) thereof as the polymorphic Form A.

Another object of the present invention is to provide a pharmaceutical composition of Zolpidem tartrate comprised of stable polymorphic Form A that does not convert to other polymorphic forms (e.g. Form C or Form E) or cause the release of Zolpidem free base during formulation of the composition into a dosage form. The Zolpidem free base is not as water soluble as the Zolpidem tartrate and thus may not be taken up by the body in vivo thereby reducing its bioavailability. It is a further object of the present invention to provide the given pharmaceutical composition in one or more of following forms; tablet (optionally with applied film coating), a capsule or in form of mini tablets filled in capsule. The preferred form is that of a tablet and more preferably in form of a film coated tablet. The tablet can be of round or oval biconvex shape with optionally scored or debossed sides if desired. The preferred shape is round. It is a further object of the present invention to provide a tablet formulation of Zolpidem tartrate Form A in which the tablet may readily be manufactured and does not demonstrate manufacturing problems such as segregation (inhomogeneity), poor flow characteristics, capping and lamination.

The desired formulation of the given composition can be obtained by commonly used technologies: dry granulation and direct compression. Preferably it is done by means of direct compression. According to the direct compression method a manufacturing process includes the following steps:

homogenization of active substance and excipients to obtain tableting blend;

compression of tableting blend into tablets, and optionally;

film coating of tablets.

Preferably, the procedure/method of obtaining a tableting blend is performed in a predetermined way that provides adequate quality in the final product in terms of homogeneity and uniformity of the tableting blend by mixing the composition of the blend under selected process parameters. The tableting blend is next processed on rotary tablet press under conditions that are selected to produce tablets of specified characteristics (e.g. appropriate hardness that ensures low friability of tablets and enables satisfactory film coating). The direct compression method preferably employed uses the least amount of operational manipulation and, in such method, the key running powder requirements (blend homogeneity, consistent bulk density, flow and compressibility) must be met by the dry blend of active agent with excipients as there is no chemical or physical modification before tabletting.

Tablets produced according to the invention can optionally be subjected to a film coating process with conventional materials used for film coating. Film coating is a process wherein tablets are tumbled in a coating pan. While the tablets are being tumbled, the film coating is applied under simultaneous application of heat.

According to the invention, a pharmaceutical composition is provided comprising Zolpidem or a pharmaceutically acceptable salt (preferably tartrate) thereof in the Form A. A Zolpidem tartrate Form A according to the invention is characterised, for example, by the XRPD profile shown in FIG. 1. The bottom profile shows the pattern of Zolpidem free base. The next highest profile is that of Zolpidem tartrate Form A. The next highest profile of FIG. 1 is the placebo for a finished Zolpidem tartrate tablet containing only the pharmaceutically inactive materials used in the tablets and no active Zolpidem ingredient formed by a direct compression method according to the invention. The highest profile of FIG. 1 is that of Zolpidem tartrate Form A tablets made with a direct compression process of the present invention. The Zolpidem tartrate Form A pattern conforms to that of prior reported patterns for Form A with characteristic major peaks at 6.5, 9.0 and 16.6. There are also characteristic minor peaks at 10.5, 13.5 and 24.6. All such measurements per FIG. 1 below are accurate to within about ±0.2 degrees two-theta. All such powder X-ray diffraction patterns were obtained by methods known in the art using an X-ray powder diffractometer It is therefore clear that this manufacturing process preserves Form A without causing its conversion to other Forms or to Zolpidem free base as shown by comparison of the highest profile in FIG. 1 (scan of compressed tablet containing Form A plus excipient) and the next highest profile (scan of compressed tablet containing only excipients and no Form A) demonstrating that there is less than at most 1% by weight of any form of Zolpidem other than form A in the compressed tablet corresponding to the material scanned in the highest profile of FIG. 1, i.e. there are no discernible peaks corresponding to any form of Zolpidem other than form A in the highest profile of FIG. 1).

A composition according to the invention can include a filler which can be selected from one or more of the following; lactose monohydrate, lactose anhydrate, starch, sugar or sugar alcohols (such as glucose, sucrose, sorbitol, mannitol), celluloses (in powder forms of different types (e.g. microcrystalline cellulose)), and dicalcium phosphate dihydrate.

The lactose may be in the form of lactose monohydrate or lactose anhydrate, but preferably comprises lactose monohydrate. The lactose may be crystalline or amorphous. Suitably, the lactose may be spray-dried (e.g. spray dried lactose anhydrate, e.g. Pharmatose™ DCL 11). The starch may for example comprise corn starch (e.g. unmodified corn starch or alternatively pre-gelatinised corn starch). The starch may also convey some disintegrant properties to the formulation. The total amount of filler present in the final composition is typically about 50 to about 92% by weight, preferably 65 to 90% by weight.

A composition according to the invention optionally includes a binder material. The binder can be selected from one or more of the following: hydroxypropyl cellulose, hydroxypropylmethyl cellulose or other cellulose ethers and vinylpyrrolidone containing polymers. Preferably, the binder comprises hydroxypropyl cellulose. Suitably the binder material will be present in the final formulation at a concentration of about 1 to about 10% by weight, preferably 2 to 5% by weight, most preferably 3 to 4% by weight. The vinylpyrrolidone containing polymer may for example comprise polyvinylpyrrolidone alone or a mixture of polyvinylpyrrolidone and a co-polymer of vinylpyrrolidone and vinyl acetate or a co-polymer of vinylpyrrolidone and vinyl acetate alone.

Typically, the binder and the filler components combined are present in the final formulation at a concentration of about 50 to about 97% by weight, preferably 70 to 95% by weight, most preferably 80 to 93% by weight.

If desired a wetting agent can be added to the formulation in order to improve dissolution of the tablet or capsule and subsequently penetration of the liquid into the pores of the dosage forms. An exemplary wetting agent is sodium lauryl sulfate. A suitable amount of wetting agent such as sodium lauryl sulfate if added is 0.3-1% such as around 0.5% w/w.

A composition according to the invention can further include an antiadherent material. The antiadherent can be selected from one or more of the following: colloidal silicon dioxide (e.g. Aerosil™ 200) or talc. Preferably, the antiadherent comprises colloidal silicon dioxide. Antiadherent is added to the composition in order to improve the flow and packing properties of the composition. Antiadherent is typically included in an amount of about 0.1 to about 5% by weight, preferably 0.5 to 1.5% by weight.

A composition according to the invention can also include a disintegrant material. The disintegrant can be selected from one or more of the following: crospovidone (cross linked polyvinylpyrrollidone), sodium starch glycolate, croscarmellose sodium, powdered cellulose, microcrystalline cellulose or carboxymethylcellulose calcium. Preferably, the disintegrant comprises sodium starch glycolate. Disintegrant is added to the composition to enhance the disintegrating properties of the composition and thereby to accelerate dissolution in vivo, e.g. in the stomach and/or intestines of the subject. Disintegrant is preferably included in an amount of about 1 to about 10% by weight, preferably 2 to 8% by weight, most preferably 4 to 7% by weight.

A lubricant is preferably included in a compostion according to the invention. The lubricant can be selected from one or more of the following: stearic acid, metal salt stearates (magnesium stearate, zinc stearate and calcium stearate), sodium stearyl fumarate, sodium lauryl sulphate, sodium benzoate, glyceryl behenate, glyceryl monostearate, glyceryl palmitostearate, polyethylene glycol, hydrogenated vegetable oil and talc. Preferably the lubricant comprises magnesium stearate. Lubricant is typically included in an amount of about 0.1 to about 5% by weight preferably 0.5 to 1.5% by weight.

A film-forming polymer, plasticizer, colorant and vehicle are preferably included in a film-coating formulation according to the invention. The film-forming polymer can be selected from one or more of the following: hydroxypropylmethyl cellulose, hydroxypropyl cellulose, methylcellulose and polyvinyl alcohol. Preferably, the film-forming polymer comprises hydroxypropylmethyl cellulose. The plasticizer can be selected from one or more of the following: glycerin, propylene glycol and polyethylene glycol. Preferably, the plasticizer comprises polyethylene glycol.

If desired a coloring agent can be added to the formulation to provide a distinguishing colour to the eventual tablet. An exemplary coloring agent is iron oxide. A suitable amount of coloring agent such as iron oxide if added is 0.02-0.15% eg 0.02-0.1% such as around 0.07% w/w. Preferably the vehicle is water.

The following examples are set forth as examples of formulations of compositions according to the invention. The starting Zolpidem or Zolpidem tartrate component of the compositions referred to in the following examples comprises essentially pure Form A that contains less than about 1%, preferably less than about 0.5% and most preferably less than about 0.1% of other forms of Zolpidem tartrate and/or the free base. Such pure Form A starting material contains between about 2 and about 3.5% by weight of water.

The final dosage forms of the pharmaceutical compositions set forth in the following examples similarly contain essentially only pure Form A Zolpidem tartrate and more particularly contain less than about 1%, preferably less than about 0.5%, more preferably less than about 0.2% and most preferably less than about 0.1% of any other form of Zolpidem tartrate or the free base.

Most preferably, the content of water in a finally formulated pharmaceutical composition according to the invention, i.e. including excipients, is between about 2% and about 8% by weight (as determined by Karl-Fischer analysis). It has been surprisingly found that prevention of undesired conversion of the specific polymorphic form A of Zolpidem tartrate and therefore the desired stability of a final pharmaceutical composition according to the invention is achieved with a content of water of less than 8% by weight, most preferably less than about 6% by weight.

Thus, according to the invention, there is provided a pharmaceutical composition comprising Zolpidem or a pharmaceutically acceptable salt thereof (preferably tartrate) in the form of Form A together with excipients, having a content of water of less than about 8% by weight (determined by Karl-Fischer analysis).

The starting pure Form A, Zolpidem tartrate material can be prepared by a variety of known methods for synthesis of Form A. One method of synthesis is by initial formation of the free base by reaction of Zolpidic acyl or acid chloride with dimethyl amine, and then subsequent reaction of the free base with tartaric acid to form a crude form of Zolpidem hemitatrate. Pure Form A can be prepared for example by crystallization of crude hemitartrate from a solution of methanol, e.g. more specifically by suspension of a selected amount of the crude hemitartrate in a solution of a selected amount of methanol at a selected temperature between about 40 and about 49 degrees Centigrade and for a selected period of time sufficient to dissolve the hemitartate material completely in solution. The so formed solution of hemitartrate is then concentrated and cooled to a temperature sufficient to cause the hemitartrate to crystallize as a solid material out of the cooled solution. The solid material so crystallized is filtered from the solution and dried, typically under vacuum and with mild heating, so as to remove excess solvent. The methanol solvent can contain from about 0.1% to about 1% water.

In the following examples, the solid materials to be formed are described as tablets that are formed by direct compression. Alternatively, a solid drug containing material for administration to a patient according to the invention, e.g. in the form of a tablet, can alternatively be formed by dry granulation as described below and used as granules or placed into capsules for administration to a subject.

In all preferred methods of formulation according to the invention, the solid drug containing materials are formed by input of less than about 20 kN compression force or equivalent pressure. As noted above, in addition to tablet forms, the solid materials formed according to the invention can be in the form of a capsule, or in the form of mini tablets filled into a capsule.

In a dry granulation method, a powdered mixture of Form A and selected excipients is first prepared, the powdered mixture having a particle size up to a maximum of about 300 μm. Granulation of the powdered mixture is carried out without moistening of the powdered drug mixture but rather by compacting large masses of the powder mixture (producing large tablets or compacts) and subsequently crushing and sizing these compacts into smaller granules, having a particle size up to a maximum of about 1200 μm.

A dry granulation method/manufacturing process according to the invention includes the following steps:

homogenization of active substance and excipients to obtain powder mixture, with particle size distribution up to 300 μm;

convert the powder mixture into large compacts by compaction/compression;

crushing and screening the compacts to produce free-flowing granules with particle size distribution up to 1200 μm;

blending granules with other excipients to obtain tableting blend;

compression of tableting blend of granules with excipients into tablets or filling of capsules with the blend of 1200 μm granules plus excipients, and, optionally film coating of tablets.

Direct compression according to the invention has the advantage of employing the least amount of operational manipulation compared to granulation technology, the key running powder requirements (blend homogeneity, consistent bulk density, flow and compressibility) must be met by the dry blend of active agent with excipients as there is no chemical or physical modification before tabletting.

A direct compression method/manufacturing process according to the invention includes the following steps:

homogenization of active substance and excipients to obtain tableting blend having a particle size of up to a maximum of about 300 μm;

compression of tableting blend into tablets at a maximum force (or equivalent pressure) of about 15 kN, preferably applying between about 8 and 20 kN of force or equivalent pressure to the blend and, optionally;

coating of tablets with an outer film.

### EXAMPLE 1

| COMPOSITION OF A TABLET | mg/tbl |
|---|---|
| Zolpidem tartrate | 10.00 |
| Starch | 21.40 |
| Lactose monohydrate | 85.00 |
| Sodium starch glycolate | 1.20 |
| Sodium lauryl sulfate | 0.60 |
| Colloidal silicon dioxide | 0.90 |
| Magnesium stearate | 0.90 |
| Lactose monohydrate | 3.00 |
| Hydroxypropylmethyl cellulose | 2.00 |
| Polyethylene glycole | 1.00 |
| Titanium dioxide | 2.00 |
| Iron oxide yellow | 0.50 |

### Preparation of Tablets:

Zolpidem tartrate, was mixed with starch, lactose monohydrate, sodium starch glycolate, sodium lauryl sulfate and colloidal silicon dioxide and homogenized in tumble type blender for 15 minutes. The blend was sieved using screening mill with conus type sieve for dry sieving.

Magnesium stearate, screened through a 0.6 mm sieve, was added to the core component above. The final blend was homogenized (tumbler blender) for additional 5 minutes and then compressed (rotary tablet press machine) into tablets. The main pressure in tableting process was within the range from 8 to 20 kN and machine speed was within 15 to 95 rpm

### Coating:

The tablets were coated using a perforated pan coater with an aqueous suspension of lactose monohydrate, hydroxypropylmethyl cellulose, polyethylene glycol, titanium dioxide and iron oxide yellow.

### EXAMPLE 2

| COMPOSITION OF A TABLET | mg/tbl |
|---|---|
| Zolpidem tartrate | 10.00 |
| Starch | 21.00 |
| Lactose monohydrate | 84.00 |
| Sodium starch glycolate | 2.00 |
| Sodium lauryl sulfate | 0.60 |
| Colloidal silicon dioxide | 0.90 |
| Magnesium stearate | 0.90 |
| Lactose monohydrate | 3.00 |
| Hydroxypropylmethyl cellulose | 2.00 |
| Polyethylene glycole | 1.00 |
| Titanium dioxide | 2.00 |
| Iron oxide yellow | 0.50 |

### Preparation of Tablets:

Zolpidem tartrate, was mixed with starch, lactose monohydrate, sodium starch glycolate, sodium lauryl sulfate and colloidal silicon dioxide and homogenized in tumble type blender for 15 minutes. The blend was sieved using screening mill with conus type sieve for dry sieving.

Magnesium stearate, screened through a 0.6 mm sieve, was added to the core component above. The final blend was homogenized (tumbler blender) for additional 5 minutes and then compressed (rotary tablet press machine) into tablets. The main pressure in tabletting process was within the range from 8 to 20 kN and machine speed was within 15 to 95 rpm.

### Coating:

The tablets were coated using perforated pan coater with aqueous suspension of lactose monohydrate, hydroxypropylmethyl cellulose, polyethylene glycol, titanium dioxide and iron oxide yellow.

### EXAMPLE 3

| COMPOSITION OF A TABLET | mg/tbl |
|---|---|
| Zolpidem tartrate | 10.00 |
| Starch | 15.00 |
| Lactose monohydrate | 88.00 |
| Sodium starch glycolate | 5.00 |
| Colloidal silicon dioxide | 1.20 |
| Magnesium stearate | 1.20 |
| Lactose monohydrate | 3.00 |
| Hydroxypropylmethyl cellulose | 2.00 |
| Polyethylene glycole | 1.00 |
| Titanium dioxide | 2.00 |
| Iron oxide yellow | 0.50 |

### Preparation of Tablets:

Zolpidem tartrate, was mixed with starch, lactose monohydrate, sodium starch glycolate and colloidal silicon dioxide and homogenized in tumble type blender for 25 minutes. The blend was sieved using screening mill with conus type sieve for dry sieving.

Magnesium stearate, screened through a 0.6 mm sieve, was added to the core component above. The final blend was homogenized (tumbler blender) for additional 5 minutes and then compressed (rotary tablet press machine) into tablets. The main pressure in tabletting process was within the range from 8 to 20 kN and machine speed was within 15 to 95 rpm

### Coating:

The tablets were coated using perforated pan coater with aqueous suspension of lactose monohydrate, hydroxypropylmethyl cellulose, polyethylene glycol, titanium dioxide and iron oxide yellow.

### EXAMPLE 4

| COMPOSITION OF A TABLET | mg/tbl |
|---|---|
| Zolpidem tartrate | 10.00 |
| Starch | 15.00 |
| Lactose monohydrate | 86.00 |
| Sodium starch glycolate | 7.00 |
| Colloidal silicon dioxide | 1.20 |
| Magnesium stearate | 1.20 |
| Lactose monohydrate | 3.00 |
| Hydroxypropylmethyl cellulose | 2.00 |
| Polyethylene glycole | 1.00 |
| Titanium dioxide | 2.00 |
| Iron oxide yellow | 0.50 |

### Preparation of Tablets:

Zolpidem tartrate, was mixed with starch, lactose monohydrate, sodium starch glycolate and colloidal silicon dioxide and homogenized in tumble type blender for 25 minutes. The blend was sieved using screening mill with conus type sieve for dry sieving.

Magnesium stearate, screened through a 0.6 mm sieve, was added to the core component above. The final blend was homogenized (tumbler blender) for additional 5 minutes and then compressed (rotary tablet press machine) into tablets. The main pressure in tabletting process was within the range from 8 to 20 kN and machine speed was within 15 to 95 rpm

### Coating:

The tablets were coated using perforated pan coater with aqueous suspension of lactose monohydrate, hydroxypropylmethyl cellulose, polyethylene glycol, titanium dioxide and iron oxide yellow.

What is claimed is:

1. A pharmaceutical composition comprising a zolpidem hemitartrate starting material: comprised of form A, the composition containing less than about 8% by weight of water.

2. The pharmaceutical composition of claim 1 wherein the composition contains less than about 1% by weight of forms of Zolpidem tartate other than form A.

3. The pharmaceutical composition of claim 1 wherein the composition is formed in a water free environment from a powdered starting material having a maximum particle size of less than about 300 μm.

4. The pharmaceutical composition of claim 1 wherein the composition is formed in a water free environment into a tablet from a powdered starting material by application of compression force of less than about 20 kN to the powdered starting material.

5. The pharmaceutical composition of claim 4 wherein the powdered starting material has a maximum particle size of less than about 300 μm.

6. The pharmaceutical composition of claim 1 wherein the Zolpidem hemitartrate starting material contains between about 2% and about 3.5% by weight of water.

7. The pharmaceutical composition of claim 1 wherein the composition contains less than about 1% by weight of forms of Zolpidem hemitartrate other than Form A.

8. The pharmaceutical composition of claim 1 wherein the composition contains less than about 0.5% by weight of forms of Zolpidem hemitartrate other than Form A.

9. The pharmaceutical composition of claim 1 wherein the composition contains less than about 0.1% by weight of forms of Zolpidem hemitartrate other than Form A.

10. The pharmaceutical composition of claim 1 wherein the composition comprises from about 50% to about 92% by weight of a filler comprised of one or more of lactose monohydrate, lactose anhydrate, starch, sugar, sugar alcohols and celluloses.

11. The pharmaceutical composition of claim 1 wherein the composition comprises from about 1% to about 10% of a binder material comprised of one or more of hydroxypropyl cellulose, hydroxypropylmethyl cellulose, cellulose ethers and vinylpyrrolidone containing polymers.

12. The pharmaceutical composition of claim 10 wherein the composition further comprises from about 1% to about 10% of a binder material comprised of one or more of hydroxypropyl cellulose, hydroxypropylmethyl cellulose, cellulose ethers and vinylpyrrolidone containing polymers.

13. The pharmaceutical composition of claim 12 wherein the composition comprises from about 50% to about 97% by weight of the filler material and the binder material combined.

14. The pharmaceutical composition of claim 1 wherein the composition has an X-Ray powder diffraction pattern having major peaks at 6.5, 9.0 and 16.6±0.2 degrees two-theta.

15. The pharmaceutical composition of claim 4 wherein the tablet is coated with a film comprising a polymer.

16. The pharmaceutical compostion of claim 15 wherein the polymer comprises one or more of hydroxypropylmethyl cellulose, hydroxypropyl cellulose, methylcellulose and polyvinyl alcohol.

17. The pharmaceutical composition of claim 15 wherein the film further comprises a plasticizer.

18. The pharmaceutical composition of claim 17 wherein the plasticizer comprises one or more of glycerin, propylene glycol and polyethylene glycol.

19. The pharmaceutical composition of claim 15 wherein the film further comprises a vehicle for components of the film.

20. The pharmaceutical composition of claim 15 wherein the film further comprises a coloring agent.

21. The pharmaceutical composition of claim 1 wherein the composition further comprises a wetting agent in amount of between about 0.3% and about 1% by weight of the composition.

22. The pharmaceutical composition of claim 1 wherein the composition further comprises an anti-adherent material in amount of between about 0.1% and about 5% by weight of the composition.

23. The pharmaceutical composition of claim 1 wherein the composition further comprises a disintegrant material in amount of between about 1% and about 10% by weight of the composition.

24. The pharmaceutical composition of claim 1 wherein the composition further comprises a lubricant material in amount of between about 0.1% and about 5% by weight of the composition.

25. The pharmaceutical composition of claim 3 wherein the powdered material is formed in a water free environment into granules having a maximum particle size of less than about 1200 μm.

26. A pharmaceutical composition comprising a Zolpidem hemitartrate starting material comprised of form A, the composition containing less than about 1% by weight of forms of Zolpidem tartate other than form A.

27. The pharmaceutical composition of claim 26 wherein the composition contains less than about 8% by weight of water.

28. The pharmaceutical compostion of claim 26 wherein the composition is formed in a water free environment into a tablet from a powdered starting material by application of compression force of less than about 20 kN to the powdered starting material.

29. A pharmaceutical composition comprising a Zolpidem hemitartrate starting material comprised of form A, the composition being formed in a water free environment into a tablet from a powdered starting material by application of compression force of less than about 20 kN to the powdered starting material.

30. The pharmaceutical composition of claim 29 wherein the composition contains less than about 8% by weight of water.

31. The pharmaceutical composition of claim 29 wherein the composition contains less than about 1% by weight of forms of Zolpidem hemitartate other than form A.

32. A pharmaceutical composition comprising a Zolpidem hemitartrate starting material comprised of form A, the composition being formed into a powdered material, the powdered material being formed in a water free environment into granules having a maximum particle size of less than about 1200 μm.

33. A pharmaceutical composition formed from a Zolpidem hemitartate starting material comprised of form A that is comprised of between about 2% and about 3.5% water.

34. The pharmaceutical composition of claim 33 wherein the composition comprises less than about 8% by weight of water.

35. The pharmaceutical composition of claim 33 wherein the composition contains less than about 1% by weight of forms of Zolpidem hemitartrate other than form A.

36. The pharmaceutical composition of claim 33 wherein the composition the composition is formed in a water free environment into a tablet from a powdered starting material by application of compression force of less than about 20 kN to the powdered starting material.

37. A method of forming a pharmaceutical composition comprising stable Form A Zolpidem hemitartrate, the method comprising the steps of:
forming a composition containing the Form A into a powdered starting material;
compressing the powdered starting material in a water free environment into a tablet by application of a pressure of less than about 20 kN to the powdered starting material.

38. The method of claim 37 wherein:
the step of forming comprises homogenizing a blend of the Form A and selected excipients to obtain tableting blend having a particle size of up to a maximum of about 300 μm.

39. The method of claim 37 further comprising the step of coating the tablet with a film comprising a polymer.

40. A method of forming a pharmaceutical composition comprising stable Form A Zolpidem hemitartrate, the method comprising the steps of:
forming a composition containing the Form A into a powdered starting material;
converting the powdered starting material in a water free environment into granules.

41. The method of claim 40 further comprising the step of compressing the granules in a water free environment into a tablet by application of a pressure of less than about 20 kN to the granules.

42. The method of claim 40 wherein the powdered starting material has a particle size of up to a maximum of about 300 μm.

43. The method of claim 40 wherein the granules have a particle size of up to a maximum of about 1200 μm.

44. The method of claim 40 further comprising encapsulating the granules in digestable capsules.

45. The method of claim 41 further comprising the step of coating the tablet with a film comprising a polymer.
